(19) United States
(12) Patent Application Publication
(10) Pub. No.: US 2019/0295725 A1
(43) Pub. Date: Sep. 26, 2019

(54) Patient Tracking and Diagnosis System of Transmissible Disease

(71) Applicant: Donnie R. Morrow, JR., Houston, TX (US)

(72) Inventor: Donnie R. Morrow, JR., Houston, TX (US)

(21) Appl. No.: 15/933,386

(22) Filed: Mar. 23, 2018

(51) Int. Cl. G16H 50/30; G16H 50/70; G01S 5/00

(52) U.S. Cl. CPC G16H 50/30; G16H 50/80; G01S 5/0027; G16H 50/70

(57) ABSTRACT

A patient tracking and diagnosis system is disclosed for providing a physician, medical diagnosis information related to a patient suspected with transmissible diseases based on the geo-location of the patient. In specific, the system comprises a mobile application module accessed by a patient to record patient location information, health information and the like, a data collection and analysis storage unit to collect, store and analyze the recorded data of various patients, a physician application module to access the patient's information and retrieve medical diagnosis information based on the geo-locations of the patient, proximity from other patients with suspected/conformed diagnosis and thereof. The invention is advantageous in aiding physicians to get knowledge of suspected diagnosis for a patient and thereby performing only those treatment tests that are necessary and thus eliminate unnecessary tests.

### FIELD OF INVENTION

The present invention generally relates to a system and method that utilizes uploaded geographical location of a patient and correlates proximity data from other patients to diagnose and predict suspected transmissible diseases, the patient might have been exposed.

### BACKGROUND OF THE INVENTION

Transmissible diseases are contagious diseases that are transmitted from one person to another or from animals to people. The communicable diseases affect people of all ages. Due to the environmental exposure children support the spread. The spread of viral diseases include influenza, measles, malaria and Ebola and other types of microorganisms are such as fungi. The types of illness include pneumonia, diarrhea, meningitis, or other clinical syndromes.

Transmissible diseases may spread in any location in different countries. It is not an easy task to track the spread of disease that take place in various locations. There is a difficulty in collecting and tracking the disease information as well as quickly communicating the disease information to prevent the spread of diseases. One of the core functions of public health surveillance is to monitor public health status and to recognize at the earliest possible time.

The communicable disease affected people in various locations will take the diagnosis at their nearest medical provider. So, the medical providers of different locations will diagnose the same treatment for the same disease affected people by providing similar tests as well as the medical prescription. There is a need of for reducing this medical waste by performing only the necessary tests and avoiding unnecessary tests. The process of tracking the communicable disease will give medical providers the knowledge of the likely disease a patient is exposed to and with the same symptoms of the same communicable disease, another patient may also be suspected by the other medical provider in a different location. The medical provider can provide the most accurate course of treatment before confirming the diagnosis with additional tests. This helps in predicting a disease and directing the patient to seek proper treatment.

There is a need for a system and method that provides faster detection and notification of the spread of disease. A recorded medical data of infected people will help in tracking the disease in a particular location. There is a need of a system that uploads the tracking data and helps in correlating the data of proximity to a confirmed disease being carried by another person. So, there will be a scope of providing the treatment for the correct disease as early as possible.

In certain cases, medical provider may have a hard time making a decision on a patient's diagnosis or may want to consider input from another medical provider who has treated patients suffering from the same disease or may have treated similar diagnosis. There is a need for an interface that facilitates medical providers to interact with one another.

In another scenario, patients who need medical attention are taken care by family members, guardians and the like. When the patient goes out it is often a concern for the members about their safety and also want to be assured that they are safe. One way a person can be assured of another person's safety is by being aware where the other person is at a given time. This boosts their confidence that they can rush to the person's location in case of emergency. However, such a provision is not provided by any of the prior art patents. Therefore, by the patient's approval, there is a need to provide the whereabouts of the patient's location to the trusted members.

In another aspect, especially after a natural or man-made disaster, the chances for outbreak of transmissible diseases are always high. There might be people to be relocated to extreme distances from their home and may likely be introduced to unsuspected diagnosis in unexpected locations. It is usually difficult to identify patients with transmissible diseases in such cases and also the time for diagnosis might be very slow. The identification of the disease involves various unnecessary tests to be performed to each and every patient who are prone to the same transmissible disease. Moreover, performing multiple tests results in the delay of treatment of the patient.

In view of above observations, there is a need for a system and method which provides faster detection and notification of a spread of disease. There is a need of a system that uploads the tracking data and helps in correlating the data of the person's proximity to a confirmed disease that being carried by another person. So, there will be a scope of providing the treatment for the correct disease as early as possible. There is a need for an interface that facilitates medical providers to interact with one another. Therefore, there is a need to provide the patient's location to the trusted members approved by the patient. There is a need for a system that aids in detecting suspected transmissible diseases in a given location based on patient's data. Further, there is a requirement to avoid performing unnecessary tests for the patients who are prone to the same transmissible disease, effected by other patients who have undergone all the required tests. Thus, there is a need to treat the correct disease as early as possible by reducing unnecessary tests.

### SUMMARY OF THE INVENTION

The invention proposes a patient tracking and diagnosis system of transmissible disease. The following presents a simplified summary in order to provide a basic understanding of some aspects of the claimed subject matter. This summary is not an extensive overview. It is not intended to identify key/critical elements or to delineate the scope of the claimed subject matter. Its sole purpose is to present some concepts in a simplified form as a prelude to the more detailed description that is presented later.

The present invention discloses a patient tracking and diagnosis system, comprising a data collection and analysis storage unit for recording, correlating, analyzing and predicting exposure of a patient to a transmissible disease based on plurality of parameters, a mobile application module with a patient interface unit that displays information and facilitates interaction of a patient with plurality of modules, the plurality of modules comprising, an entry module for accessing plurality of modules, for registration of a patient through proper credentials by the patient and a receiving means for entry into the system, a location module for recoding at least a geo-location from a patient device, a monitoring unit that records data related to patient's health and connected to the patient device, a data storage module for storing information entered by the patient and recorded by the modules of the said mobile application module, a communicator to receive and transmit information to a data collection analysis storage unit, a patient notification module to receive notifications from physicians, medical authorities, pharmaceutical companies and the like, wherein the notifications may be related to the field of medicine, follow-ups, motivational, updates and thereof.

The system of the present invention comprises a physician application module with a physician interface unit that displays information and facilitates interaction of a physician with plurality of modules, the plurality of modules comprising an entry module for accessing plurality of modules, for registration of a physician through proper credentials by the physician and a receiving means for entry into the system, a retrieval module for entering of a search criteria related to at least a patient by the physician and thereby retrieve information such as location of the patient, medical history, suspected diagnosis, confirmed diagnosis of other patients proximity of the patient from other patients and thereof, an update module to receive updated diagnosis, misdiagnosis of the physician by comparing the diagnosis data with other physicians for a same transmissible disease, a peer to peer module for requesting medical information from another physician, follow up diagnosis and thereof and a physician notification module which facilitates the physician to send notifications to the treated patients, their contacts and thereof.

The present invention discloses A method of generating medical diagnosis information of a patient, based on geo-location, comprising the steps of receiving of search query related to a patient by a data collection and analysis storage unit entered by a physician through a retrieval module, wherein the search query can include input such as patient ID, health insurance number, location, address, suspected diagnosis and the like, identifying data stored in the data collection and analysis storage unit related to the received search query, correlating the search query with the data of plurality of other patients suspected or confirmed to have transmissible disease, wherein said data of plurality of other patients includes geo-locations matched with the patients recorded geo-locations, proximity of the patient from other patients, patients monitoring unit data and the like, generating a prediction analysis report displaying list of suggested diagnosis, proximity of the patient with confirmed diagnosis and or suspected diagnosis patients and thereof, receiving at least a diagnosis selected by the physician from the list of suggested diagnosis and thereby displaying suggested treatment and/or notes on the effectiveness of a given treatment, saving the medical status of the entered patient, initially with the respective suspected diagnosis selected by the physician and receiving updates by the physician, regarding the updated diagnosis, medical tests, misdiagnosis of the patient treatment and thereof, wherein the physician said can further send notifications to patients, guardians and the like.

The present invention A method of updating the physician related to the diagnosis for a patient, comprises of recording various types of transmissible disease diagnosed by plurality of physicians, identifying the suspected diagnosis treated by a physician, identifying diagnosis tests performed by plurality of physicians for the respective suspected diagnosis treated by a physician, correlating the diagnosis tests performed by plurality of physicians for the respective suspected diagnosis treated by a physician with the suspected diagnosis tests for the same transmissible disease and notifying the physician of misdiagnosis if the physician hasn't diagnosed the patient with treatment test/tests performed by at least another physician and confirmed diagnosis, updating the physician by notifying new diagnosis tests and thereof.

### DESCRIPTION OF DRAWINGS

The accompanying drawings, which are incorporated in and constitute a part of the specification, illustrate an embodiment of the invention, and, together with the description, serve to explain the principles of the invention.

FIG. 1 illustrates a block diagram of a patient tracking and diagnosis system in accordance to the invention.

FIG. 2A illustrates an exemplary process for registration of patients with the mobile application module in accordance to the invention.

FIG. 2B illustrates an exemplary process for registration of physicians with the physician application module in accordance to the invention.

FIG. 3 illustrates a flowchart for transmission of data from mobile application module to the data collection and analysis storage unit.

FIG. 4 illustrates a flowchart for data correlation and predictive analysis performed by the data collection and analysis storage unit in accordance to the invention.

FIG. 5 illustrates a flowchart of search feature of the physician application module in accordance to the invention.

FIG. 6 illustrates a flowchart for a method of updating the physician related to diagnosis for a patient in accordance to the invention.

FIG. 7 illustrates a flowchart to manage and track the peer to peer information request in accordance to the invention.

### DETAILED DESCRIPTION OF DRAWINGS

An exemplary embodiment of the present invention will be described in reference to the accompanying drawings. Wherever possible, same or similar reference numerals are used in the drawings and the description to refer to the same or like parts or steps.

The term transmissible disease refers to a contagious disease is a subset category of transmissible diseases, which are transmitted to other persons, either by physical contact with the person suffering the disease, or by casual contact with their secretions or objects touched by them or airborne route among the other routes. The term physician includes doctors, medical providers and the like health specialists.

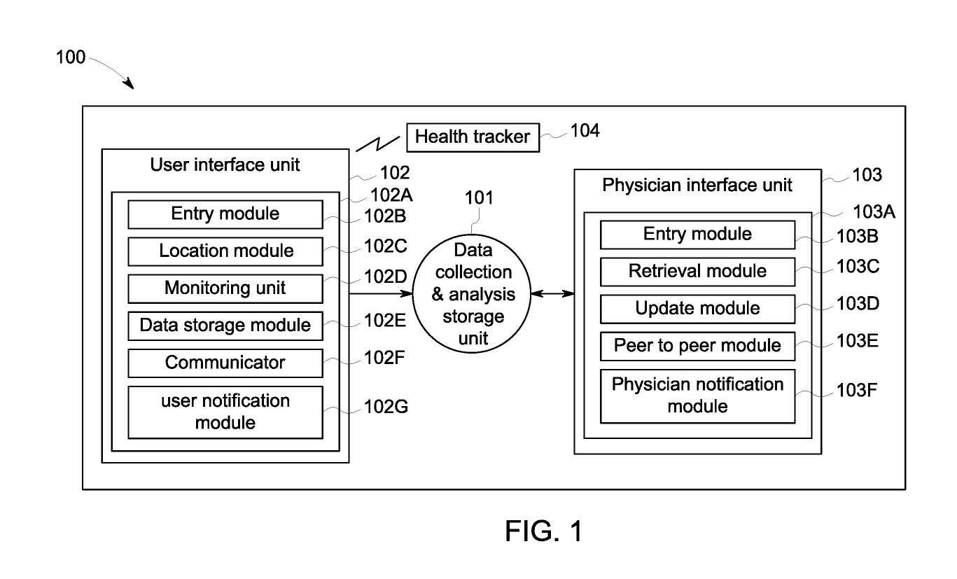

The invention describes a system 100 which utilizes geo-location of a patient and related medical records to correlate the proximity data with other patients to diagnose and predict suspected transmissible disease(s) the patient might have been exposed. As illustrated in FIG. 1, a patient tracking and diagnosis system 100 comprises a data collection and analysis storage unit 101 for recording, correlating, analyzing and predicting exposure of a patient to a transmissible disease based on plurality of parameters, a mobile application module 102, a physician application module 103, a monitoring unit 104 that records data related to patient's health and communicates to the personal device of the patient which in turn is recorded by the mobile application module. In accordance to the invention, the patient tracking and diagnosis system 100 facilitates physicians to suspect transmissible disease that a patient might be exposed, based on the patients with suspected or confirmed transmissible disease in the recorded location history of the patient and thereby suggests the corresponding diagnosis tests. In accordance to an exemplary embodiment of the invention, the mobile application module 102 comprises a patient interface unit 102A to display information and facilitate patients to interact with the plurality of modules such as an entry module 102B for registration of patient with the mobile application module and accessing the mobile application module 102, location unit 102C for recording of at least a geo-location a patient has been while time stamping the geo-locations through a time keeper, monitoring unit 102D which may be an external health tracker to track the patient's health condition and to communicate the information to the personal device with the mobile application module 102, data storage module 102E to store information of location, credentials, time stamping and thereof, a communicator 102F to transfer and receive information from the data collection and analysis storage unit 101 and a patient notification module 102G to notify the patient of the information received from the data collection and analysis storage unit 101.

In accordance to an exemplary embodiment of the invention, the data collection and analysis storage unit 101 receives information from the mobile application module 102, wherein said information includes location history of the respective patient, time stamping of the location, health condition of the patient and thereof. The data collection and analysis storage unit 101 stores the received information from multiple patients, correlates the information and thereby generates a predictive analysis report of the transmissible disease that the patient might have contacted the suspected or confirmed patients with the transmissible disease(s). The invention is effective in suggesting the physician, a diagnosis test to be performed by displaying the physicians, data of patients with transmissible disease(s) in the locations the patient had been to and thereby the respective test to detect if the patient has the same transmissible disease.

According to an embodiment of the invention, the system 100 comprise a physician application module 103 to facilitate the physician to search and retrieve a list of suggested diagnosis compiled by the data collection and analysis storage unit 101. In accordance to the invention, data collection and analysis storage unit 101 may be a storage unit but not limited to a server, a cloud network, database and the like networks that serve the purpose of being recited. As illustrated in FIG. 1, the physician application module 103 comprises a physician interface unit 103A, an entry module 103B for registration of the physician with the system 100, a retrieval module to enter patient credentials and thereby retrieve diagnosis data, an update module 103C for updating the medical diagnosis status of a patient, a peer to peer module 103D to interact with at least a physician related to the diagnosis and thereof and a physician notification module 103E to receive notifications from the data collection and analysis storage unit 101 of misdiagnosis, new treatment tests, changed patient diagnosis and thereof and also sends notifications to patients by the physician.

According to an embodiment of the invention, the mobile application module collects patient's health data such as body temperature, heart rate, physical exertion, blood pressure and thereof recorded by the monitoring unit 104. The monitoring unit may be a health tracker connected to the patient device with mobile application module. The patient device may be a smart phone, iphone, handheld device and thereof. In one embodiment of the invention, the monitoring unit may be a gadget worn by the patient such as wristband, activity tracker, smart watch, etc. The monitoring unit may be wirelessly connected to the patient device. Further the communication between the monitoring unit and the patient device may be any communication means known to the person skilled in the art.

In accordance to an exemplary embodiment of the invention, the patients of the mobile application module need to register with the system to access the mobile application module.

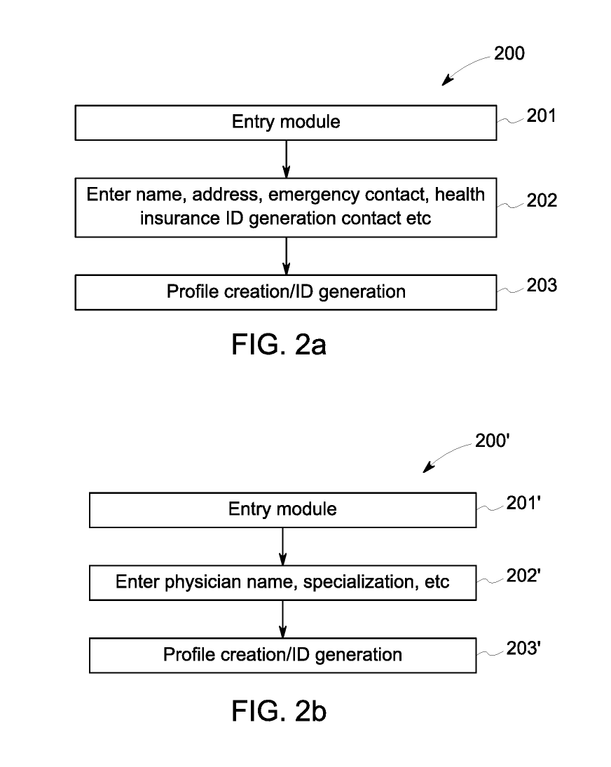

FIG. 2A illustrates an exemplary process for registration of patients 200. As illustrated, at step 201 the patient selects the entry module displayed on the interface. At step 202 the patient is requested to enter his/her name, address, health insurance ID, emergency contact, guardian contact and the like information. In accordance to the invention, emergency contacts are those contacts that any person the patient would approve to help him makes decisions related to the health and wellbeing of the patient, guardian contacts are any person the patient would require to help him makes decisions related to the health and wellbeing of the patient. The mobile application module may allow the patient to set configurations for the service on the device and to allow the patient to disable display advertisements. At step 203, upon entry of information by the patient, a profile is created and the patient is allotted with a login name and password to enter into the entry module of the mobile application module. The registration process may include any process known by a person skilled in the art.

FIG. 2B illustrates an exemplary process for registration of physicians 200′. As illustrated, at step 201 the physician selects the entry module displayed on the physician interface unit. At step 202 the physician is requested to enter name, address, specialization, medical practice location, name and the like information. At step 203, upon entry of information, a profile is created and the physician is allotted with a username and password to enter into the entry module of the physician application module. The registration process may include any process known by a person skilled in the art.

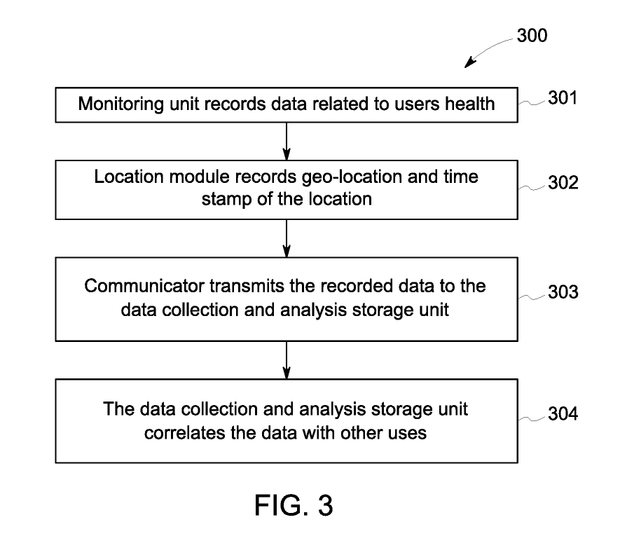

According to the invention, the information recorded by the mobile application module is transmitted to the data collection and analysis storage unit to be stored. FIG. 3 illustrates flowchart of transmission of data from mobile application module to the data collection and analysis storage unit 300. At step 301, the monitoring unit records the patient's health data such as body temperature, heart rate, physical exertion, blood pressure and thereof. The health data may be recorded from the patient's device, worn devices and from any device having information medically related to the patient which is connected to the patient's device. As in step 302, the location module records the geo-location data and time stamp of the location. The location module records location history of the patient, proximity and thereof and stores in the data storage module. At step 303, the recorded data collected from the monitoring unit and the location module is transmitted to the data collection and analysis storage unit by means of a communicator. In accordance to the invention, the data transmission may be scheduled by the patient. In accordance to the invention, the data transmission may be through a push activated by the patient. In accordance to the invention, the data transmission may take place upon receiving a push request from the server. At step 304, the data collection and analysis storage unit receives the transmitted data from the mobile application modules of patient devices. In an exemplary embodiment of the invention, the transmitted data may be stored in an indexed database. At step 304, the transmitted data is stored, analyzed, correlated with location data, health data and the like data of other patients.

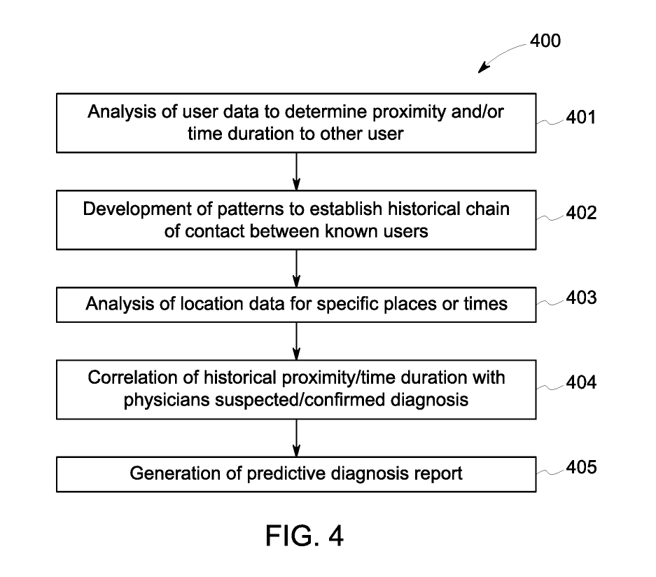

In accordance to the invention, the data collection and analysis storage unit performs data correlation and predictive analysis as illustrated as a flowchart 400 in FIG. 4. At step 401, the system uses predetermined algorithms to determine proximity and/or time duration of the patient with other patients. In an aspect of the invention, the proximity data may be divided into multiple categories defined as close proximity, general proximity and geographical proximity. The time duration may be divided into multiple categories defined as a short, medium and long. In accordance to an exemplary aspect, the proximity and time duration may also be inferred. For example, patient A and patient B are identified at location A. Later, patient A and patient B may be found at location B. This could be inferred that patient A and patient B have traveled together (through some transportation means) and Proximity data was either lost or misleading during a given time frame.

In accordance to the invention, as in step 402, the system even develops patterns to establish historical chain of contacts between known patients. The patterns will identify patient A coming into contact with patient B who comes in contact with patient C and so forth. According to the invention, as in step 403, the data collection and analysis storage unit also analyzes location data for specific places or times (hot spots). It may be identified that patients who sought medical care had visited a common location with or without a common time frame. For example, it may be identified that 3 different patients visited the same establishment and all three were diagnosed as having a food borne illness. In another aspect of the invention, it may be identified that patients who sought medical care had visited common locations with or without a common time frame. It may be identified that several patients over a time period had received a similar diagnosis and it may be identified that they all had a common intersection point.

In accordance to the invention, as in step 404, the data collection and analysis storage unit correlates historical proximity/time duration with the physician. Suspected/confirmed diagnosis is included to provide a predictive diagnosis report for a given patient. The various categories of suspected or confirmed diagnosis are weighed. Known communicable disease knowledge maybe utilized for forming the predictive diagnosis report but should not get limited to incubation time lines, method of spreading and other data uploads. The patient may be utilized for forming the predictive diagnosis report. For example patient A and patient B both had high heart rates and had high levels of physical exertion. It may be inferred that the patients were within close proximity and may have shared one of a very specific subset of communicable diseases. As in step 405, the system generates a predictive analysis report for a given patient based on the recited steps. In accordance to the invention, the data collection and analysis storage unit utilize predictive analysis to back track data and validate previous diagnosis for a given patient.

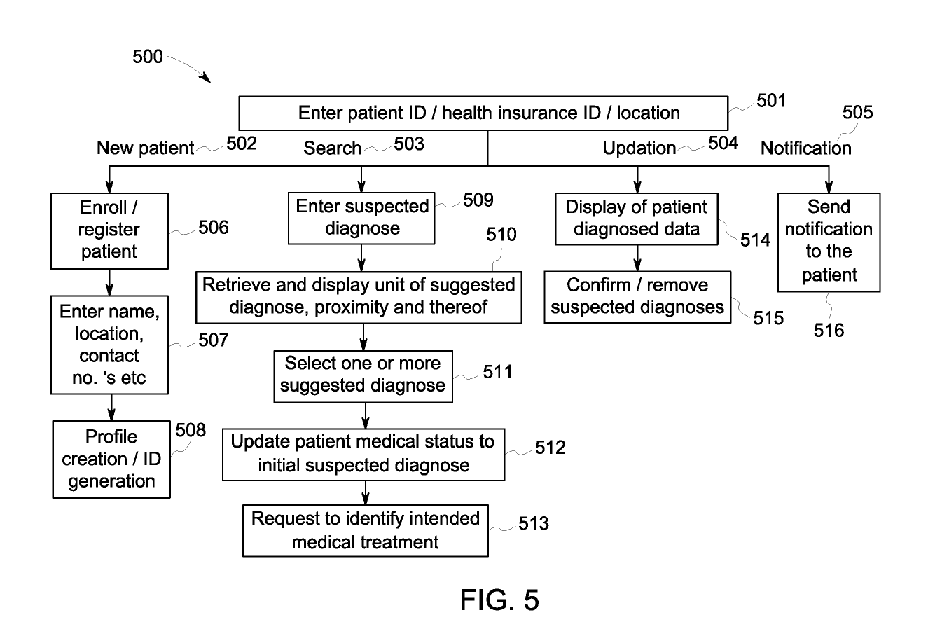

In accordance to an embodiment of the invention, the physician application module enables the physician to enter patient ID and thereby retrieves the patient related information. FIG. 5 illustrates flowchart 500 of search feature of the physician application module. At step 501, the physician selects the retrieval module and enters patient ID and/or health insurance ID and/or location of the patient to retrieve information from the data collection and analysis storage unit. As in step 502, the physician can enroll/register patients who haven't registered with the system. As in step 503, the physician can search about a patient and retrieve information stored in data collection and analysis storage unit. As in step 504, physician can select updates to view updates received related to diagnosis of the patient. As in step 505, the physician can send notifications to patients and/or to the contacts related to the patient and may receive updates related to new treatments or misdiagnosis of a patient, based on the updated diagnosis by other physicians.

According to an embodiment of the invention the physician can enroll/register new patients/patients to the system in the event the patients didn't get registered themselves with the mobile application module of the system. At step 506, the physician selects an option in the physician application module to enter/enroll new patients into the system. At step 507, the physician is requested to enter name of the patient, address, health insurance ID, emergency contact, guardian contact and the like information. A request may be sent from the physician application module to a patient mobile application asking for access. In accordance to the invention, the physician can enter multiple alternative addresses of the patient, such as the patient's home address, work address or any location that the patient believes they may have come in contact with a transmissible disease. In accordance to the invention, mapping tool maybe provided to the physician to locate addresses or geographical locations of the patient. The physician can draw a polygon on the map to provide a specific area for inquiry. The entered patient information, addresses, geo location and thereof will be recorded by the data collection and analysis storage unit. At step 508, a profile is created and a patient ID and password are generated for entry of the patient into the system.

In accordance to the invention, the physician can enter a patient ID to retrieve information stored in the system related to the patient through the retrieval module of the physician application module. The physician can enter any of the patient information such as patient ID, health insurance ID, address of the patient etc. As in step 509, the physician is further requested to enter suspected diagnosis, a patient might be suspected of getting affected by examination of the symptoms of the patient. In a key aspect of the invention, the search may not actually use these suspected diagnosis but if the physician application module tags the patient then that is being searched as suspected of having the respective suspected diagnosis. Upon entry of the recited patient information, the retrieval module displays results showing a list of suggested diagnosis compiled in the data collection and analysis storage unit, i.e. the patients with suspected or confirmed with a transmissible disease in the locations of the patient, the proximity of the patient with said patients at various times and places and thereof. In accordance to the invention, the results will be divided into multiple categories defined as Diagnosis Confirmation wherein said category represents the patients with suspected transmissible disease and patients who are confirmed to have the transmissible disease and Proximity wherein the category displays that range of distance the patient has come in contact with patients suspected to have transmissible disease or patients who have transmissible disease. The proximity may be displayed as close, general and geographical. At step 511, the physician is requested to select one or more suggested diagnosis from the results displayed. At step 512, the patient's medical status is changed initially as suspected with the suggested diagnosis selected by the physician and is stored/updated in the data collection and analysis storage unit. At step 513, the physician may be requested to identify the intended medical treatment. The physician may also be provided with a suggested treatment and/or notes on the effectiveness of a given treatment, which may be stored, for the treatment and/or notes from other physicians who have treated similar diagnosis. Physicians may or may not be selected from the patients related to the given patient.

According to another embodiment of the invention, the physician can access previous data of the patient to update the medical status of the patient as in step 504. The physician upon entering the patient's ID, health insurance ID and thereof is displayed with the patient information but not limited only to the medical status of the patient, treatment procedure, previous suspected/confirmed diagnosis identified, previous suspected/confirmed diagnosis identified by a physician, previous noted treatments, previous prescriptions, a heat map of patient's locations, charts, graphs and/or data points related to data acquired through the patient's mobile application system. The display of patient diagnosed data depicted at step 514. As another aspect of the invention, the medical status of the patient can be changed by the physician, based on the current status of the patient as confirmed to have a suspected diagnose which is depicted at step 515. The physician can also remove the patient confirmation diagnosis status to indicate that the patient is no longer affected with the transmissible disease.

According to another embodiment of the invention, the physician may follow-up a patient who is being treated. As in step 505, the physician may send notifications to patients and/or guardians of patients which is depicted in 516. In an example not limited to, the physician may send to request treatment follow-ups, to provide motivational information, to provide or request for general medical information and a request to get updated worn device and/or other device data.

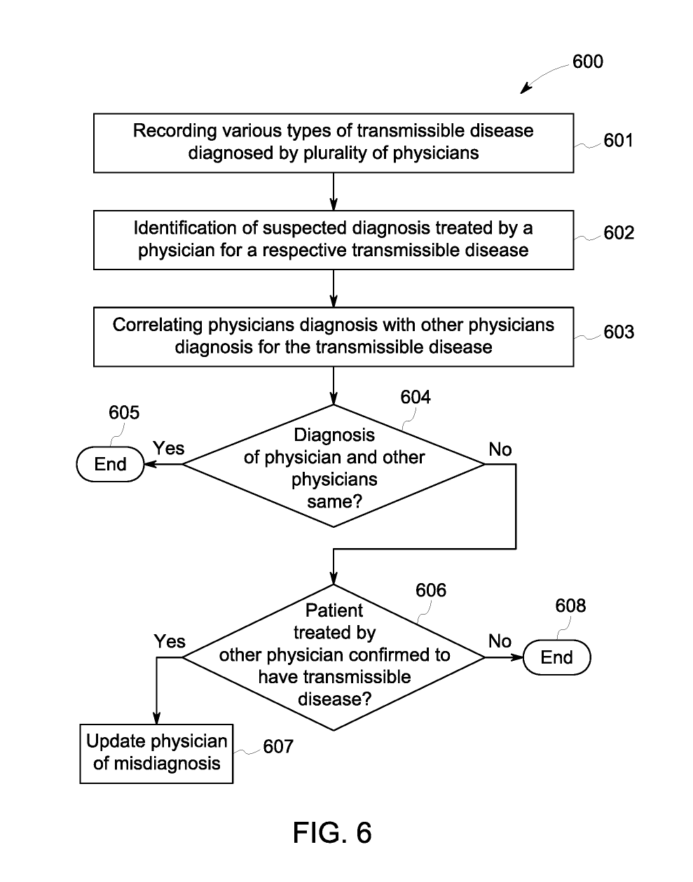

According to an embodiment, the physician also receives updates related misdiagnosis, updated diagnosis performed by other physicians and thereof. FIG. 6 illustrates flowchart 600 for a method of updating the physician related to the diagnosis for a patient. At step 601, the data collection and analysis unit records collection of various types of transmissible disease diagnosed by plurality of physicians. At step 602, suspected diagnosis treated by a physician for a respective transmissible disease is identified. At step 603, the diagnosis tests performed by plurality of physicians for the respective suspected diagnosis treated by a physician is correlated with the suspected diagnosis tests for the respective transmissible disease. At step 604, a data collection and analysis storage unit evaluates if the physician diagnosis and other physicians diagnosis for the same type of transmissible disease is same. If the diagnosis of both physicians is same, the logic ends as in step 605. However as in step 606, if the diagnosis of both physicians is not same, the logic determines whether or not the patient treated by other physician is confirmed to have transmissible disease. If the patient treated by other physician is found to have transmissible diseases and the physician haven't diagnose the patient as confirmed to have the transmissible disease, the physician is updated that the patient has been misdiagnosed as depicted in step 607. If the patient treated by other physician is found to have transmissible diseases and the physician have diagnosed the patient as confirmed to have the transmissible disease, then the process ends at step 608.

According to an embodiment of the invention, the physician can select the peer to peer module to follow up at least another physicians conclusions and treatment, request information and thereof. In certain cases, medical provider may have a hard time making a decision on a patient's diagnosis or may want to consider input from another medical provider who has treated patients related to their patient or have treated similar diagnosis. The peer to peer module will facilitate sending the request through the data collection and analysis storage unit to the best identifiable medical providers. The peer to peer module displays responses to their requests. The peer to peer module displays the request for responses.

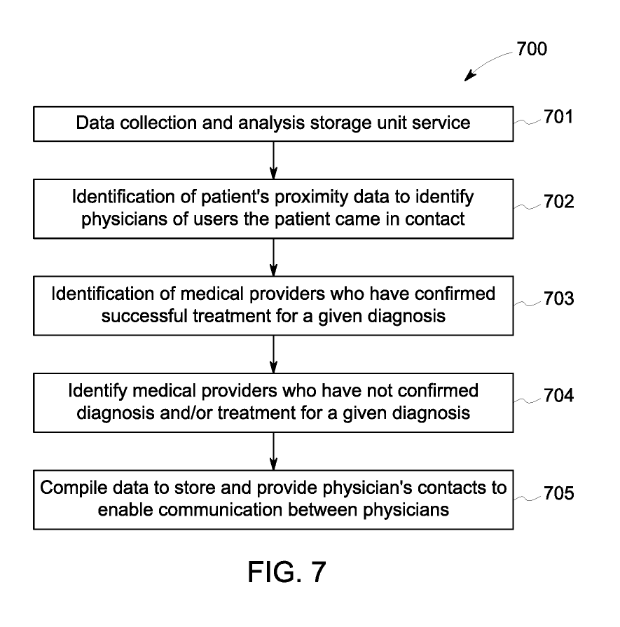

In accordance to the invention, the data collection and analysis storage unit can manage and track the peer to peer information request as illustrated in flowchart 700 of FIG. 7. The data collection and analysis storage unit may identify physicians to contact based on the data processed by the Data correlation and predictive analysis at step 701. At step 702, the patient's proximity data is identified to identify physicians the patients have contacted for treatment. At step 703, the analysis identifies medical providers who have confirmed successful treatment for a given diagnosis. At step 704, the analysis identifies medical providers who have not confirmed diagnosis and/or treatment for a given diagnosis. At step 705, the recited data is compiled to provide physician's contacts in order to enable communication between physicians, example when requested by a physician accepting and accessing the peer to peer module. This may be useful in comparing notes on an unexpected/unknown disease.

In accordance to an exemplary embodiment, the invention facilitates guardians and/or persons who have recorded as emergency contacts by a patient to locate the patient. This may be performed only upon approval of the patient. The guardians and/or persons who have recorded as emergency contacts by a patient can track the patient by obtaining exact location data, providing exact historical location data and thereof. In accordance to another aspect of the invention, the system may collect any medical related information from a health insurance provider such as for example but not limited to confirmation of currently suspected diagnosis a suspected/Confirmed Diagnosis not already stored in the system. The system may send any contractually obligated data to the Health Insurance provider.

It is to be understood that the invention is advantageous in providing heat maps of communicable diseases from a high level geographic area down to the street level. The system will also be able to produce predictive heat maps for communicable diseases using various predictive algorithms.

The invention can be applicable to pharmacy companies. Pharmaceutical companies will be provided upon request with approval a report for a requested area. The types of diagnosis/treatments that medical providers are pursuing will give them real time feedback on the type of pharmaceuticals and/or supplies they should be stocking both by current data and predictive data. Pharmaceutical companies may utilize the system with approval to initiate a physician outreach using various levels of data analysis for various reasons including but not limited to updates related to their products and/or advertisements for their products. Further the physicians' medical Service companies may utilize this service with approval to initiate a physician outreach using various levels of data analysis for various reasons including but not limited to updates related to their products and advertisements for their products.

The invention can be applicable to reach public in creating an awareness of the transmissible diseases in their location. A proper medical authority may utilize this service with approval to initiate a patient outreach using various levels of data analysis for various reasons including but not limited to conduct drives for immunizations, public notifications about disease prevention, disease response, suggested activities and precautions directed at high-risk populations. In another aspect, a pharmaceutical company or medical provider service may use the system with approval to update patients on information regarding their products or services.

A proper medical authority may utilize this service with approval to initiate a Medical Provider outreach using various levels of data analysis for various reasons including but not limited to peer to peer information request about suggested treatments, additional patient outreach, medical coordination requests and thereof.

The system is applicable in addressing of disaster response. The system will be able to identify patients who have been relocated to temporary shelters in the event of a disaster by means of the location data recorded by the location module of the system. The system will be able to track the spread of disease outbreaks within these confined areas. The system will be accessible to medical and supply coordinators to have real time feedback for providing the services where the medical personnel and supplies need to be located. The system will continue to track the patients and diseases as they disperse from the temporary shelters. Thus, the system avoids performing unnecessary tests for the patients who are prone to the same transmissible disease, effected by other patients who have undergone all the required tests. Therefore, the system allows physicians to treat correct disease as early as possible by reducing unnecessary tests.

It is to be understood, that the location data of the patient may be identified through the global position system (GPS), cellular triangulation, Wi-Fi location and/or triangulation, RFID location and/or triangulation, written/typed location and any technology serving the purpose of the invention.

It is to be understood that the order in which the steps or system described is not intended to be construed as a limitation, and any number of the described steps can be combined in any order to implement the method, or an alternative method. Additionally, individual steps may be deleted from the method without departing from the spirit and scope of the subject matter described herein. It will be understood that even though the system and method is described with reference to the computing system, the description may be extended to other systems as well.

I claim:

1. A patient tracking and diagnosis system, comprising:
a data collection and analysis storage unit for recording, correlating, analyzing and predicting exposure of a patient to a transmissible disease, based on plurality of parameters;
a mobile application module with a patient interface unit that displays information and facilitates interaction of a patient with plurality of modules, wherein said plurality of modules comprising:
an entry module for accessing plurality of modules, for registration of the patient through proper credentials and a receiving means for entry into the system;
a location module for recoding at least a geo-location from a patient device;
a monitoring unit connected to the patient device that records the data related to patient's health;
a data storage module for storing of information entered by the patient and being recorded by the modules of said mobile application module;
a communicator to receive and transmit information to said data collection and analysis storage unit;
a patient notification module to receive notifications from physicians, medical authorities, pharmaceutical companies and the like, wherein the notifications may be related to the field of medicine, follow-ups, motivational, updates and thereof;
a physician application module with a physician interface unit that displays information and facilitates interaction of a physician with plurality of modules, the plurality of modules comprising:
an entry module for accessing plurality of modules, for registration of a physician through proper credentials and a receiving means for entry into the system;
a retrieval module for entering a search criteria related to at least one patient and thereby retrieving information such as location of the patient, medical history, previous suspected or confirmed diagnosis, and thereof;
an update module to receive updated diagnosis, misdiagnosis of the physician by comparing diagnosis data with other physicians for same transmissible disease;
a peer to peer module to request medical information, follow up diagnosis and thereof from another physician; and
a physician notification module to send notifications to treated patients, their contacts and thereof.

2. The patient tracking and diagnosis system as claimed in claim 1, wherein said data collection and analysis storage unit receives data from plurality of patients, stores and correlates the data with at least one patient and thereby generates a suggested transmissible disease diagnosis prediction analysis report, wherein said prediction analysis report is generated based on list of suspected and/or confirmed diagnosis patients, proximity of the patients and thereof.

3. The patient tracking and diagnosis system as claimed in claim 1, wherein said plurality of parameters include geo-location, location history, time stamping of geo-location, data related to patient's health such as body temperature, heart rate, physical exertion and thereof; and also the suspected and/or confirmed patient's with transmissible disease(s), proximity of the patient from other patients and thereof.

4. The patient tracking and diagnosis system as claimed in claim 1, wherein said data collection and analysis storage unit receives data in different locations from plurality of sources, wherein said plurality of sources include various physicians who medically treated patients, patient who sought medical care, data from respective monitoring unit of at least one patient and thereof.

5. The patient tracking and diagnosis system as claimed in claim 1, wherein said data collection and analysis storage unit may be a database, a cloud network, a server and the like.

6. The patient tracking and diagnosis system as claimed in claim 1, wherein said location module further includes a time keeper for time stamping the geo-locations of the patient.

7. The patient tracking and diagnosis system as claimed in claim 1, wherein said retrieval module stores the entered patient medical status by the physician as patient suspected with transmissible disease, wherein said retrieval module can be accessed by the physician to retrieve patient information such as medical status and thereby changes the medical status of the patient from suspected diagnosis to confirmed diagnosis or removes the confirmed diagnosis.

8. The patient tracking and diagnosis system as claimed in claim 1, wherein said update module updates physicians of misdiagnosis, new diagnosis and thereof of a patient by correlating the diagnosis data of various physicians for a respective transmissible disease.

9. The patient tracking and diagnosis system as claimed in claim 1, wherein said physician can access said peer to peer module for contacting other physicians to request information, to follow-up and to review another physician's medical conclusions, treatments and thereof.

10. A method of generating medical diagnosis information of a patient based on geo-location, comprises the steps of:
receiving of search query related to a patient by a data collection and analysis storage unit entered by a physician through a retrieval module, wherein the search query can include input such as patient ID, health insurance number, location, address, suspected diagnosis and the like;
identifying data stored in the data collection and analysis storage unit relating to the received search query;
correlating the search query with data of plurality of other patients who are suspected or confirmed to have transmissible disease, wherein said data of plurality of other patients include geo-locations matched with patient's recorded geo-location, proximity of the patient from other patients and the like;
generating a prediction analysis report and displaying the list of suggested diagnosis, proximity of the patient with confirmed diagnosis and or suspected diagnosis patients and thereof;
receiving at least one diagnosis selected by the physician from the list of suggested diagnosis and thereby displaying suggested treatment and/or notes on the effectiveness of a given treatment;
saving medical status of the entered patient initially with respective suspected diagnosis selected by the physician; and
receiving updates by the physician related to updated diagnosis, medical tests, and misdiagnosis of the patient treatment and thereof, wherein said physician can further send notifications to patients, guardians and the like.

11. The method of generating medical diagnosis information of a patient based on geo-location as claimed in claim 10, wherein said suggested diagnosis is based on suspected or confirmed transmissible disease of patients who are proximate to the patient, patients in the geo-location of the patient and the like.

12. The method of generating medical diagnosis information of a patient based on geo-location as claimed in claim 10, wherein said prediction analysis report of the system may include analyzing the patient data to determine proximity and/or time duration from other patient(s), developing patterns to establish a historical chain of contacts between known patients, analyzing location data for specific places or times the patient had been to, correlating historical proximity/time duration with physicians suspected/confirmed diagnosis; and generating of a predictive diagnosis report for a given patient.

13. The method of generating medical diagnosis information of a patient as claimed in claim 10, wherein said prediction analysis report is performed by data collection and analysis storage unit and displayed to a physician upon request by the physician through the physician application module.

14. A method of updating the physicians related to diagnosis of a patient, comprises:
recording various types of transmissible diseases, diagnosed by plurality of physicians;
identifying suspected diagnosis provided by the physician;
identifying diagnosis tests performed by plurality of physicians for respective suspected diagnosis;
correlating the diagnosis tests performed by plurality of physicians with the suspected diagnosis tests for same transmissible disease;
notifying the physician of misdiagnosis if the physician hasn't diagnosed the patient with diagnosis tests performed by at least another physician; and
updating the physician by notifying new diagnosis tests and thereof.

15. The method of updating physician related to diagnosis of a patient as claimed in claim 14, wherein plurality of patients' diagnosis performed by said plurality of physicians for the same transmissible disease is recorded and compared by the system.

16. The method of updating physician related to diagnosis of a patient as claimed in claim 14, wherein said diagnosis of one physician varies with diagnosis of another physician for the same transmissible disease, wherein said variation may be in terms of a diagnosis test performed by one physician for a patient and confirming to have a transmissible disease and wherein the diagnosis test is not performed by other physician, the respective another physician updates that misdiagnosis has been performed.

17. The method of updating physician related to diagnosis of a patient as claimed in claim 14, wherein said method further notifies the physician of a suggested diagnosis for a patient with the comparing diagnosis of other physicians for the same transmissible disease.

18. The method of updating physician related to diagnosis of a patient as claimed in claim 14, wherein said notification may be categorized as urgent, wherein meaning that there was a high degree of certainty of a misdiagnosis and if said notification is categorized as suggested wherein meaning that the search results would return back to a different top diagnosis.
